(19) United States
(12) Patent Application Publication
(10) Pub. No.: US 2010/0273239 A1
(43) Pub. Date: Oct. 28, 2010

(54) LACTOBACILLUS HELVETICUS BACTERIUM COMPOSITION FOR TREATMENT OF ATOPIC DERMATITIS

(76) Inventor: Benedicte Flambard, Frederiksberg (DK)

Correspondence Address: FOLEY AND LARDNER LLP;SUITE 500, 3000 K STREET NW, WASHINGTON, DC, 20007, US

(21) Appl. No.: 12/664,963

(22) PCT Filed: Jun. 26, 2008
(86) PCT No.: PCT/EP08/58134

§ 371 Date: Jun. 14, 2010

(30) Foreign Application Priority Data
Jun. 27, 2007 (DK) PA 2007 00932

(51) Int. Cl. C12N 1/20

(52) U.S. Cl. 435/252.9

(57) ABSTRACT

A Lactobacillus helveticus bacterium composition for treatment of atopic dermatitis in a human.

### FIELD OF INVENTION

The present invention relates a Lactobacillus helveticus bacterium composition for treatment of atopic dermatitis of humans.

### TECHNICAL BACKGROUND AND PRIOR ART

Atopic dermatitis (AD) is a chronic inflammatory itching skin disease that affects a large number of children and adults in industrialized countries.

AD is treated in a number of ways such as by use of antihistamines, topical or systemic steroids. See e.g. the 2006 review article in “Allergy 2006: 61: 969-987”.

It has also been described to use different lactic acid bacteria (LAB) for treatment of different allergy problems such as AD.

US2006/0088513A1 provides a summary of the art with respect to this.

The relevant parts of the description of US2006/0088513A1 are discussed below.

It is publicly known that L. rhamnosus GG strain is an anti-allergic lactic acid bacteria and it is reported that by administering it to expectant mothers, the development of atopic dermatitis of the child is suppressed.

The use of the lactic acid bacteria as an anti-allergic drug is disclosed in several prior art documents. As examples of disclosure the use of lactic acid bacteria such as L. acidophilus, L. brevis, L. buchnerii, L. casei and Lactobacillus leuteri, as inhibitors of type I allergy such as allergic bronchial asthma, chronic allergic rhinitis, atopic dermatitis and the like can be mentioned.

Recently, a lot of research in the direction of trying to suppress allergy by special food/drink products has been carried out. As a result, anti-allergic effects are described/published in relation to special ingredients of various foods or drinks such as Japanese basil oil, fish oil, particular tea polyphenol and the like. Some lactic acid bacteria fermented yogurts or lactic acid bacteria beverages are in practice used as foods or drinks said to having anti-allergic effect.

In prior art such as in US2006/0088513A1 it is normally described that the LAB shall preferably be administered as live/viable LAB cells in order to get the positive relevant anti-allergy effect.

However, in WO02/28402 (Nestlé) it is described that LAB can also be given as dead/deactivated cells or as so-called LAB “culture supernatants” [comprising relevant metabolites(s)] and still give some improvement of skin related allergy reactions such as e.g. atopic dermatitis.

As explained in section [0006] of US2006/0088513A1, it is not appropriate to say that all LAB have a relevant anti-allergy effect and it is therefore still important to identify/select new useful LAB strains.

### SUMMARY OF THE INVENTION

The problem to be solved by the present invention is to identify an alternative lactic acid bacterium (LAB) that can be used to make a food/feed product or a medicament for treatment of atopic dermatitis (AD).

The solution is based on that the present inventors further analyzed the special Lactobacillus helveticus bacterium with the registration/accession number DSM 14998 as described in WO2004/015125 and WO2006/125441 (both have Chr. Hansen A/S as applicant). This LAB with registration number DSM 14998 may herein also be termed “Cardi-04®” or “CHCC5951”.

In WO2004/015125 and WO2006/125441 it is described that Cardi-04® has good blood pressure reducing properties but it is not described nor is anything suggested about atopic dermatitis effects of Cardi-04®.

As can be seen in working example 1 herein the inventors identified that the atopic dermatitis (AD) of a human adult person virtually disappeared after he orally had taken a Cardi-04® fermented milk composition. As can be seen in working example 1, the person had suffered for a long time and earlier tried to treat his AD with all kinds of medicine, natural herbs or different lotions.

In example 1 milk fermented with Cardi-04® was used. Without being limited to theory it is believed that some of the Cardi-04® metabolites/peptides have anti-AD effect. The peptides may e.g. be peptides derived from the liberation of peptides from casein in the milk by the proteolytic activity of the LAB.

However, there is objectively no reason to believe that Cardi-04® could not be administered as such, i.e. as live/viable cells to a person to get a good AD treatment. As described above, giving live/viable LAB to treat AD is widely described in the prior art.

As described above, Cardi-04® is a Lactobacillus helveticus bacterium.

Accordingly, a first aspect of the invention relates to use of a Lactobacillus helveticus bacterium for the manufacture of a product for the treatment of atopic dermatitis (AD) in a human, wherein the product comprises:
(i): live/viable or dead Lactobacillus helveticus bacteria;
(ii): Lactobacillus helveticus bacteria fermented milk; or
(iii): a mixture of both (i) and (ii).

### Definitions:

Prior to a discussion of the detailed embodiments of the invention a definition of specific terms related to the main aspect of the invention is provided.

The term “atopic dermatitis” is a well-known term for the skilled person and a doctor knows to diagnose a person to identify whether or not this person suffers from atopic dermatitis.

According to the well known Medical Encyclopedia of MedlinePlus (NIH, USA) atopic dermatitis relates to an inflammatory response of the skin, caused by contact with allergens or irritants, exposure to sunlight, or by poor circulation, even stress. An example of atopic dermatitis is atopic eczema, an itchy rash that produces redness, blisters and scaling.

Scratching the rash may spread the inflammation; lead to infection and even leave scars.

Embodiments of the present invention are described below, by way of examples only.

### DETAILED DISCLOSURE OF THE INVENTION

### Lactobacillus helveticus

A sample of the particular preferred Lactobacillus helveticus strain CHCC5951/Cardi-04® was deposited at DSMZ (Deutsche Sammlung von Mikroorganismen and Zellkulturen GmbH, Mascheroder Weg 1b, 38124 Braunschweig, Germany) under the accession number DSM 14998 with a deposit date of 15 May 2002. The deposit has been made under the conditions of the Budapest Treaty on the International Recognition of the Deposit of Microorganisms for the Purposes of Patent Procedure.

Accordingly, in a preferred embodiment the Lactobacillus helveticus bacterium is the Lactobacillus helveticus bacterium with the registration number DSM 14998.

### Product

The product may be any food or pharmaceutical product, or a cosmetic product for oral or topical application. Examples of food or pharmaceutical products are milk, yogurt, curd, cheese, fermented milks, milk based fermented products, ice-creams, fermented cereal based products, milk based powders, infant formulae, pet food, tablets, liquid bacterial suspensions, dried oral supplement, wet oral supplement, dry tube-feeding or wet tube-feeding. For cosmetic products lotions, shampoos, creams, such as moisturizing creams, sun-screens, after-sun creams or anti-aging creams, and/or ointments are envisaged, wherein the bacteria may be included in a live form, semi-active or in deactivated form, e. g. as a lyophilized powder.

Product—Comprising Live/Viable or Dead Lactobacillus helveticus Bacteria

As said above the product may be a product comprising live/viable or dead Lactobacillus helveticus bacteria in accordance with point (i) of first aspects.

As known to the skilled person (see e.g. WO02/28402) when administered to an individual human as bacteria the probiotic's balancing activity will be dose dependant. Thus, it is envisaged to include as many as from 10⁵ to 10¹² bacteria/g product, in which the bacteria may be alive or dead. Preferably, they are live/viable.

Product—Comprising Lactobacillus helveticus Bacteria Fermented Milk

As said above the product may be a product comprising Lactobacillus helveticus bacteria fermented milk in accordance with point (ii) of first aspects.

The skilled person knows how to make LAB fermentation of milk. Reference is made to e.g. WO2004/015125 describing LAB milk fermentation in details.

The use of lactic acid bacteria as described herein provides a useful amount of peptides or other active components with good AD reducing properties directly after fermentation. Consequently, it may not be necessary to further purify or up-concentrate the peptides or other active components from the fermented milk. The fermented food milk may be packed directly and provided to the market as e.g. a food product, preferably a functional food product, or a food product additive, e.g. in a freeze-dried form.

Further freeze-dried fermented milk could be suspended in neutral milk and thereby give a suitable food product. The freeze-dried fermented milk could therefore be seen as a suitable food additive product.

The term “functional food” denotes herein a food product where the consumer in some way is informed that it has a useful function in relation to AD reducing properties.

In some circumstances it may be preferred to perform a subsequent purification of the peptides or other active components from the fermented food milk. This may for instance be when the peptides or other active components are to be used in a medicament such as a pharmaceutical tablet, which requires a very high concentration of the AD reducing peptides or other active components. Accordingly, in an embodiment of the invention the product is a medicament.

An embodiment of the invention relates to a use as described herein where the fermented milk is further processed in a way that purifies or up-concentrates the peptides or other active components with AD reducing properties.

A suitable way of making the up-concentration is where the fermented milk, containing peptides or other active components with AD reducing properties, is centrifuged, and the resulting supernatant comprising the peptides or other active components are recovered.

When fermenting milk such a supernatant composition is termed whey. As can be seen in example 1, whey is a product with excellent AD reducing properties. Accordingly in a preferred embodiment the product/composition is whey.

The centrifugation may preferably be performed, for example, at 2,000 to 20,000 rpm for 1 to 20 minutes. The centrifugation may also be performed in a centrifuge.

The resulting supernatant may be subjected to further purifying treatment with a reverse-phase resin, for obtaining a sample in which the content of the peptides or other active components with heart rate reducing properties is increased. The purifying treatment with a reverse-phase resin may be performed by absorption and elution of the peptides or other active components with a reverse-phase resin, and/or by reverse-phase chromatography, thereby increasing purity of the peptides or other active components.

As shown in working example 1 herein the atopic dermatitis (AD) of a human adult person virtually disappeared after he orally had taken a Cardi-04® fermented milk composition. As explained in example 1, the fermented milk product was given to the male adult person in a volume of 150 ml/day for a period of 8 weeks.

As known to the skilled person a pharmaceutically relevant dosage to get the wanted medical effect may vary from person to person and also vary according to the severeness of the actual disease to be treated.

Accordingly, in an embodiment of the invention the fermented milk is given to the human person in a volume of from 5 ml/day to 1000 ml/day, more preferably from 25 ml/day to 500 ml/day. Preferably, in is given for a period from 1 day to 1 year, more preferably from 1 week to 25 weeks.

### Human

The human may be a child or an adult. In a preferred embodiment it is an adult.

### EXAMPLES

### Example 1

Human Person Treated for AD with Fermented Milk

The Lactobacillus helveticus bacterium used to ferment milk was Cardi-04®. The fermented milk and subsequent centrifugation to get whey were made as described in the working examples of WO2004/015125.

This fermented milk product was given to a male adult person in a volume of 150 ml/day for a period of 8 weeks.

The result was summarized by the test person in a letter to inventors of the present patent application. Overall the letter said:

I'm participating in your Cardi-04® research project and have noticed something very special. Since childhood I have suffered from atopic eczema—and have tried to cure it with all kinds of medicine, natural herbs, different lotions and more. BUT—during the last couple of weeks I have had close to no trouble with eczema. I have experienced no change in lifestyle or eating habits—apart from participating in your research project.

### CONCLUSION

This result demonstrated that Cardi-04® has very good anti-allergy effect to thereby make an efficient treatment of atopic eczema.

### REFERENCES

1: Allergy 2006: 61: 969-987
2: US2006/0088513A1
3: WO02/28402 (Nestlé)
4: WO2004/015125 (Chr. Hansen A/S)
5: WO2006/125441 (Chr. Hansen A/S)

1. Use of a Lactobacillus helveticus bacterium for the manufacture of a product for the treatment of atopic dermatitis (AD) in a human, wherein the product comprises:
(i): live/viable or dead Lactobacillus helveticus bacteria;
(ii): Lactobacillus helveticus bacteria fermented milk; or
(iii): a mixture of both (i) and (ii).

2. The use according to claim 1, wherein the atopic dermatitis (AD) is atopic eczema.

3. The use according to claim 1, wherein the Lactobacillus helveticus bacterium is the Lactobacillus helveticus bacterium with the registration number DSM 14998 (herein also termed “Cardi-04”).

4. The use according to claim 1, wherein the product is a food, a pharmaceutical or a cosmetic product for oral or topical application.

5. The use according to claim 4, wherein
the food product or pharmaceutical product is milk, yogurt, curd, cheese, fermented milks, milk based fermented products, ice-creams, fermented cereal based products, milk based powders, infant formulae or pet food, or tablets, liquid bacterial suspensions, dried oral supplement, wet oral supplement, dry tube-feeding or wet tube-feeding; or
the cosmetic product is lotion, shampoo, cream.

6. The use according to claim 1, wherein the product is a food product (e.g. yogurt) and it comprises Lactobacillus helveticus bacteria fermented milk.

7. The use according to claim 6, wherein the fermented milk is whey.

8. The use according to claim 6, wherein the fermented milk is freeze-dried and the product is a food additive product.

9. The use according to claim 6, wherein the product comprises from 10⁵ to 10¹² live/viable bacteria/g product.

10. The use according to claim 1, wherein the human is a child or an adult, preferably wherein the human is an adult.
